(19) United States
(12) Patent Application Publication
(10) Pub. No.: US 2009/0029394 A1
(43) Pub. Date: Jan. 29, 2009

(54) Novel Assay for the Detection of an Antibody Bound to a Cell Membrane Receptor

(76) Inventor: Timo Piironen, Ilmarinen (FI)

Correspondence Address: BIRCH STEWART KOLASCH & BIRCH, PO BOX 747, FALLS CHURCH, VA, 22040-0747, US

(21) Appl. No.: 12/224,554

(22) PCT Filed: Mar. 6, 2007
(86) PCT No.: PCT/FI2007/050120

§ 371 Date: Aug. 29, 2008

(30) Foreign Application Priority Data
Mar. 7, 2006 (FI) 20065152

(51) Int. Cl. G01N 33/53

(52) U.S. Cl. 435/7.24

(57) ABSTRACT

This invention relates to the design of a novel immunoassay specific for the measurement of humanized antibody, Campath-1H, bound to the CD52 cell membrane receptor. The method can be used for pharmacokinetic studies and for monitoring purposes. The invention reveals improvements in higher specificities and sensitivities that can be obtained in relation to the conventionally used methods.

### FIELD OF THE INVENTION

This invention relates to the design of a novel immunoassay specific for the measurement of a humanized antibody, namely Campath-1H (alemtuzumab), and to the use of the novel assay for e.g. pharmacokinetic studies and for monitoring purposes. Suitable biological specimens for the immunoassay determinations are biological fluids, e.g. serum samples. The invention reveals improvements in higher specificities and sensitivities that can be obtained in relation to the conventionally used methods.

### INTRODUCTION AND BACKGROUND

Campath-1H (alemtuzumab) is a humanized monoclonal antibody (IgG1) specific for the binding of CD52 molecule presented on cell membranes. The CD52 antigen is a lipid-anchored glycoprotein abundantly expressed on lymphocytes and a few other cell types. The mature antigen contains a protein component of only 12 amino acids. The antigenic epitope recognized by Campath-1H comprises the C-terminal amino acids together with part of the lipid anchor, which makes analytics of Campath-1H challenging. Therefore, an intact cell membrane receptor is needed for high affinity binding of Campath-1H. In addition, commonly used secondary anti-species antibody reagents cross-reacting with the excess of non-specific human antibodies in biological samples often result in poor selectivity and high variability.

Campath-1H (alemtuzumab) can cause lysis of normal and malignant lymphocytes through complement mediated cytotoxicity and antibody-dependent cell-mediated cytotoxicity. Campath-1H is being developed for the use of the treatment of chronic lymphocytic leukemia (CLL), and as immunosuppressant in transplantation, and for the treatment of autoimmune diseases.

There are only few published Campath-1H assays utilizing either cell based fluorescence activated cell sorter (FACS) analysis [Rebello and Hale, J Imm Meth 2002, 260; 285-302] or recently reported enzyme linked immunosorbent assay (ELISA) [Jilani et al., Leukemia Res 2004, 28; 1255-62], both assay methods having inadequate selectivities and sensitivities required for pharmacokinetic studies and monitoring purposes. The reported LLOQ's (lower limit of quantification) for methods described by Rebello & Hale and Jilani et al. were 0.5 μg/ml and 0.25 μg/ml, respectively. Further, in addition of having an inadequate selectivity and sensitivity, said FACS analysis is laborious and time consuming and the method by Jilani et al. has not been successfully reiterated.

### SUMMARY OF THE INVENTION AND PREFERRED EMBODIMENTS

We have designed and validated a novel competitive assay for the sensitive and specific detection of humanized antibody, Campath-1H (alemtuzumab), in human samples, based on the use of a labeled antibody and competitive assay format using commercially available filter plates.

An object of the invention is therefore a competitive method for assaying humanized antibody, Campath-1H (alemtuzumab), which binds to the CD52 cell membrane receptor, said method comprising the steps of:

(a) obtaining a sample to be analyzed for the presence of the antibody;

(b) binding CD52 receptor containing cells or cell membrane preparations to a filter plate membrane;

(c) contacting the analyte antibody, Campath-1H, labeled with a detectable label and the test sample with the filter plate membrane, thus letting the labeled antibody and the antibody in the test sample compete for binding to cell membrane receptors in the filter plate membrane;

(d) washing unbound reagents through the filter plate membrane;

(e) detecting the presence of the label and determining the amount of the analyte antibody by referring to a calibration standard curve.

A preferred competitive assay according to the invention is based on the use of Campath-1H labeled with a fluorescent label, preferably Eu.

A further object of the invention is a test kit for assaying Campath-1H (alemtuzumab), said kit comprising
a detectable label attached to the analyte antibody
a filter plate membrane
a (lyophilized or frozen) cell or cell membrane preparation containing CD52 receptor in a suitable container. If desired, the test kit may also comprise suitable buffers needed for the test.

Preferably the test kit comprises Eu-labeled Campath-1H. The filter plate membrane for use in the method and in the test kit according to the invention is for example a commercially available Acrowell 96 filter plate (Pall Life Sciences).

The invention is hereinbelow described in more detail referring to the accompanied drawings.

### BRIEF DESCRIPTION OF THE FIGURES

FIG. 1 illustrates an assay design according to the invention for the analysis of Campath-1H.

FIG. 2 is an assay calibration standard curve (mean±SD) for competitive Campath-1H assay in human serum, showing LLOQ and ULOQ (upper limit of quantification).

### DETAILED DESCRIPTION OF THE INVENTION

The antibody to be assayed by the method according to the invention is Campath-1H (alemtuzumab). However, all animal, human or humanized antibodies that bind to cell membrane receptors can be assayed by a corresponding method. Such antibodies include e.g. gemtuzumab ozogamicin (Mylotarg) and rituximab (Rituxan, MabThera), the corresponding cell membrane receptors being CD33 and CD20, respectively. Other monoclonal antibodies that can be assayed by the method according to the invention include abciximab (Reopro, Centorix), basiliximab (Simulect), bevacizumab (Avastin), cetuximab (Erbitux), daclizumab (Zenapax), efalizumab (Raptiva), infliximab (Remicade, Avakine), lintuzumab (Zamyl), natalizumab (Tysabri), omalizumab (Xolair), palivizumab (Synagis), panitumumab (Vectibix), tositumomab (Bexxar), trastuzumab (Herceptin), and other chimeric monoclonal antibodies. The biological fluid to be analysed is e.g. serum, plasma, whole blood, cerebrospinal fluid or synovial fluid sample, preferably a serum sample.

In the competitive assay method according to the present invention, cells or cell membrane preparations are bound to filter plate membranes. Cell lines expressing the required cell receptor CD52 and cell culture media are commercially available or can be prepared by methods known to persons skilled in the art. Cell membrane preparations can be prepared by homogenizing and subsequent centrifugation step by methods known to persons skilled in the art.

Suitable filter plate membranes are commercially available and include e.g. Acrowell filter plate membranes obtainable from Pall Life Sciences.

A suitable detectable label for the purposes of the invention is a fluorescent label. Alternatively, enzymatic and radioactive labels or magnetic particles may also be used, if appropriate.

Preferred fluorescent labels include all commonly used fluorescent labels, such as europium (Eu). Labelling of the antibody can be carried out e.g. by labelling free amine groups. The label is detected by using a label counter suitable for the detection of the label in question.

A calibration standard curve is provided by preparing calibration standards of the analyte antibody, by measuring the signal and fitting the data to a standard curve, preferably by using a suitable evaluation software.

In conclusion, we have established a novel methodological concept for a sensitive and specific determination of a receptor bound antibody, Campath-1H (alemtuzumab), in biological samples such as serum. More than ten-fold improvement of lower limit of quantification (LLOQ) of the assay compared to other reported assay methods of Campath-1H is achieved by using reagents of excellent technical performance in a carefully optimized assay design, as shown below. The good specificity of the Campath-1H assay especially with regard to the cross-reactivity with abundant circulating non-specific human antibodies was achieved predominantly due to a competitive assay approach (therefore not using secondary anti-human antibody reagents) and the use of filter plates.

According to a further aspect of the invention, it is most likely applied to patient samples where pharmacokinetic studies or monitoring of patients is needed.

1. Materials and Methods

1.1 Antibodies, Cell Lines, Reagents and Instrumentation

Campath-1H (alemtuzumab, MabCampath, Schering AG, Germany) 10 mg/mL infusion solution was obtained from pharmacy. T-cell lymphocyte cutaneous lymphoma cell line HuT 78 (catalog no. TIB-161) expressing CD52 receptor and cell culture media were obtained from ATCC (Manassas, Va., USA). Acrowell 96 filter plates (prod. no. 5020) were obtained from Pall Life Sciences (Ann Arbor, Mich., USA). Superdex 75 and 200 HR 10/30 FPLC and NAP-5 columns were obtained from Amersham Pharmacia Biotech (Uppsala, Sweden). The Victor multi-label counter, MultiCalc evaluation software, DELFIA Eu-labeling kit, L×R binding assay buffer, L×R wash solution and enhancement solution were obtained from Perkin-Elmer Life Sciences (Turku, Finland). MultiScreen vacuum wash manifold was obtained from Millipore (Billerica, Mass., USA).

1.2 Eu-Labeling of Campath-1H

Campath-1H was labeled with Eu-chelate to the extent of approximately 2-3 Eu/Campath-1H. Briefly, in order to remove the TRIS buffer containing amino groups capable of reacting with the later added Eu-chelate, Campath-1H antibody solution was added to the NAP-5 column and eluted with 0.05 M carbonate buffer, pH 9.8. The antibody solution was added to approximately 120-fold molar excess of N1-Eu⁺³ chelate (N¹-(p-isothiocyanatobenzyl)-diethylenetriamine-N¹,N²,N³,N³-Tetra-acetate-Eu) and incubated over night at 4° C. The Eu-labeled Campath-1H was separated from the free Eu-chelate by size exclusion chromatography using the Superdex 200 HR 10/30 column according to the instructions in DELFIA Eu-labeling kit using TSA-buffer (pH 7.8) for elution.

1.3 Assay Design

According to the invention, a novel competitive assay was designed and validated for the measurement of Campath-1H in human serum based on the use of intact cells or cell membranes, Eu-labeled Campath-1H and filter plates.

1.4 Assay Validation

1.4.1 Selectivity

Selectivity was studied testing serum pool of healthy blood donors and minimum of six individual control patients.

1.4.2 Precision and Accuracy

Intra- and inter-assay precision and accuracy was evaluated by analyzing five different quality control sample concentrations prepared in human serum matrix in six replicate measurements (each measurement was a mean of duplicate results) conducted over several days by two different analysts (total of three assays).

1.4.3 Lower and Upper Limit of Quantification

Lower limit of quantification (LLOQ) was determined as a lowest quality control level with precision and accuracy below 25% and 75-125%, respectively. Upper limit of quantification (ULOQ) was determined as a highest quality control level with precision and accuracy below 20% and 80-120%, respectively.

2 Results

2.1 Assay Design

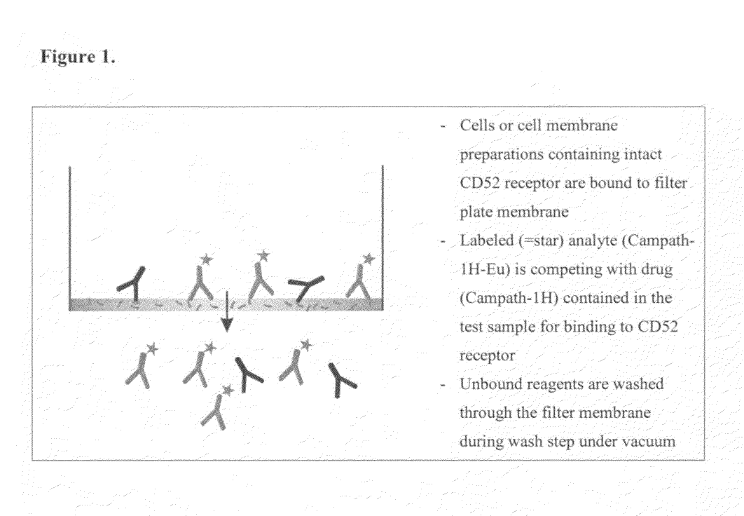

Diagram of the assay design is shown in FIG. 1.

2.1.1 Cell Culture and Preparation of Cell Membranes

T-cell lymphocyte cutaneous lymphoma cell line HuT 78 expressing CD52 receptor was grown in solution (Iscove's Modified Dulbecco's Medium +20% fetal bovine serum). Membrane stocks were prepared in ice-cold TME-buffer (50 mM Tris-HCl, 10 mM MgCl₂ and 1 mM EDTA) and homogenized using bead mill homogenizer. Nuclei and unbroken cells were removed by centrifugation at approx. 220 g for 10 min at 4° C. The supernatant was centrifuged again at approx. 40000 g for 45 min at 4° C. The final pellets were suspended in TME-buffer and stored at −70° C. until use.

2.1.2 Competitive Assay

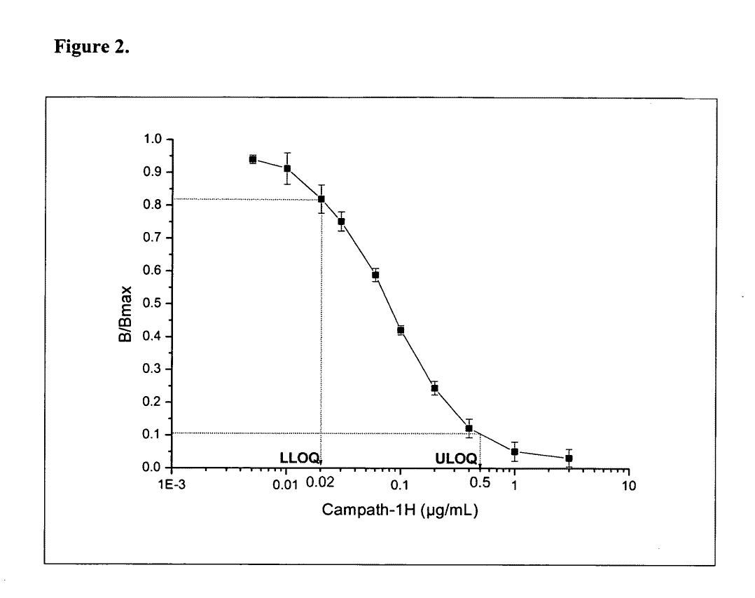

Whole cells or membrane stock preparation diluted in L×R binding buffer were added to the filter plate (50 μL/well) and incubated for 1 h at room temperature. Subsequently, calibration standards prepared in human serum ranging from 0.005 to 3 μg/mL, quality control samples prepared in human serum and study samples (30 μL/well) were added in duplicate and incubated for 2 h at room temperature. Finally, Eu-labeled Campath-1H (2.5 ng/well/50 μL) diluted in L×R binding buffer and incubated over night at 4° C. The plates were then washed six times in Millipore vacuum manifold using L×R wash buffer followed by the addition of DELFIA Enhancement Solution (200 μL/well). Fluorescence (Eu) was measured after 15 min incubation at room temperature with shaking. MultiCalc evaluation software was used for fitting the standards and creating concentration data. Calibration standard curve of three assay sets is shown in FIG. 2.

2.2 Assay Validation

2.2.1 Selectivity

All tested human control serum pools (n=2) and six tested individual healthy control samples showed concentrations below LLOQ (0.02 μg/mL).

2.2.2 Precision and Accuracy

Intra-assay precision (CV) of the method for quality control (QC) samples prepared in human serum at concentrations 0.02, 0.03, 0.05, 0.1 and 0.5 μg/mL was established to be 4.2-28.2% (Table 1). Intra-assay accuracy (AC) of the method for quality control samples prepared in human serum at concentrations 0.02, 0.03, 0.05, 0.1 and 0.5 μg/mL was established to be 88-117% (Table 1).

TABLE 1
Intra-assay precision and accuracy.
Quality control samples - intra-assay data: Campath-1H [μg/mL]

| | | | | | | | | | | | |
|---|---|---|---|---|---|---|---|---|---|---|---|
| 1 | 2 | 3 | 4 | 5 | 6 | N | Mean | SD | CV [%] | AC [%] | Bias [%] |

QC 0.02 μg/mL

| | | | | | | | | | | | | |
|---|---|---|---|---|---|---|---|---|---|---|---|---|
| Set 1 | 0.018 | 0.019 | 0.017 | [0.017] | 0.027 | 0.027 | 5 | 0.022 | 0.005 | 23.1 | 108 | 8 |
| Set 2 | 0.021 | 0.022 | 0.018 | 0.021 | 0.020 | 0.020 | 6 | 0.020 | 0.001 | 6.7 | 102 | 2 |
| Set 3 | [0.015] | 0.017 | 0.017 | 0.020 | 0.020 | 0.021 | 5 | 0.019 | 0.002 | 9.8 | 95 | −5 |

QC 0.03 μg/mL

| | | | | | | | | | | | | |
|---|---|---|---|---|---|---|---|---|---|---|---|---|
| Set 1 | 0.034 | 0.034 | 0.034 | 0.037 | [0.031] | 0.037 | 5 | 0.035 | 0.002 | 4.7 | 117 | 17 |
| Set 2 | 0.034 | 0.03 | [0.033] | 0.03 | 0.031 | 0.033 | 5 | 0.032 | 0.002 | 5.7 | 105 | 5 |
| Set 3 | [0.016] | 0.017 | 0.022 | 0.026 | 0.031 | 0.036 | 5 | 0.026 | 0.007 | 28.2 | 88 | −12 |

QC 0.05 μg/mL

| | | | | | | | | | | | | |
|---|---|---|---|---|---|---|---|---|---|---|---|---|
| Set 1 | 0.050 | 0.052 | 0.051 | 0.056 | 0.054 | 0.056 | 6 | 0.053 | 0.003 | 4.8 | 106 | 6 |
| Set 2 | 0.058 | 0.060 | 0.056 | 0.057 | 0.054 | 0.050 | 6 | 0.056 | 0.003 | 6.2 | 112 | 12 |
| Set 3 | 0.041 | 0.045 | 0.045 | 0.053 | 0.047 | 0.048 | 6 | 0.047 | 0.004 | 8.6 | 93 | −7 |

QC 0.1 μg/mL

| | | | | | | | | | | | | |
|---|---|---|---|---|---|---|---|---|---|---|---|---|
| Set 1 | 0.094 | 0.115 | 0.108 | 0.102 | 0.108 | 0.097 | 6 | 0.104 | 0.008 | 7.5 | 104 | 4 |
| Set 2 | 0.114 | 0.121 | 0.117 | 0.114 | 0.109 | 0.103 | 6 | 0.113 | 0.006 | 5.6 | 113 | 13 |
| Set 3 | 0.101 | 0.105 | 0.105 | 0.121 | 0.115 | 0.110 | 6 | 0.110 | 0.007 | 6.8 | 110 | 10 |

QC 0.5 μg/mL

| | | | | | | | | | | | | |
|---|---|---|---|---|---|---|---|---|---|---|---|---|
| Set 1 | 0.533 | 0.541 | 0.482 | 0.503 | 0.530 | 0.544 | 6 | 0.522 | 0.024 | 4.7 | 104 | 4 |
| Set 2 | 0.521 | 0.538 | 0.583 | 0.607 | 0.568 | 0.545 | 6 | 0.560 | 0.032 | 5.7 | 112 | 12 |
| Set 3 | 0.445 | 0.494 | 0.468 | 0.490 | 0.497 | 0.470 | 6 | 0.477 | 0.020 | 4.2 | 95 | −5 |

[ . . . ] = Result rejected due to CV of duplicate measurements >30%

Inter-assay precision (CV) of the method for quality control (QC) samples prepared in human serum at concentrations 0.02, 0.03, 0.05, 0.1 and 0.5 μg/mL was established to be 7.1-18.1% (Table 2). Inter-assay accuracy (AC) of the method for quality control samples prepared in human serum at concentrations 0.02, 0.03, 0.05, 0.1 and 0.5 μg/mL was established to be 102-109% (Table 2).

TABLE 2
Inter-assay precision and accuracy.
Quality control samples - inter-assay data: Campath-1H

| Set no. | QC no. | Unit | QC 0.02 | QC 0.03 | QC 0.05 | QC 0.1 | QC 0.5 |
|---|---|---|---|---|---|---|---|
| Set 1 | 1 | μg/mL | 0.018 | 0.034 | 0.050 | 0.094 | 0.533 |
| | 2 | μg/mL | 0.019 | 0.034 | 0.052 | 0.115 | 0.541 |
| | 3 | μg/mL | 0.017 | 0.034 | 0.051 | 0.108 | 0.482 |
| | 4 | μg/mL | [0.017] | 0.037 | 0.056 | 0.102 | 0.503 |
| | 5 | μg/mL | 0.027 | [0.031] | 0.054 | 0.108 | 0.530 |
| | 6 | μg/mL | 0.027 | 0.037 | 0.056 | 0.097 | 0.544 |
| Set 2 | 1 | μg/mL | 0.021 | 0.034 | 0.058 | 0.114 | 0.521 |
| | 2 | μg/mL | 0.022 | 0.030 | 0.060 | 0.121 | 0.538 |
| | 3 | μg/mL | 0.018 | [0.033] | 0.056 | 0.117 | 0.583 |
| | 4 | μg/mL | 0.021 | 0.030 | 0.057 | 0.114 | 0.607 |
| | 5 | μg/mL | 0.020 | 0.031 | 0.054 | 0.109 | 0.568 |
| | 6 | μg/mL | 0.020 | 0.033 | 0.050 | 0.103 | 0.545 |
| Set 3 | 1 | μg/mL | [0.015] | [0.016] | 0.041 | 0.101 | 0.445 |
| | 2 | μg/mL | 0.017 | 0.017 | 0.045 | 0.105 | 0.494 |
| | 3 | μg/mL | 0.017 | 0.022 | 0.045 | 0.105 | 0.468 |
| | 4 | μg/mL | 0.020 | 0.026 | 0.053 | 0.121 | 0.490 |
| | 5 | μg/mL | 0.020 | 0.031 | 0.047 | 0.115 | 0.497 |
| | 6 | μg/mL | 0.021 | 0.036 | 0.048 | 0.110 | 0.470 |
| N | | | 16 | 15 | 18 | 18 | 18 |
| Nominal concentration | | μg/mL | 0.020 | 0.030 | 0.050 | 0.100 | 0.500 |
| Experimental mean | | μg/mL | 0.020 | 0.031 | 0.052 | 0.109 | 0.520 |
| SD | | μg/mL | 0.003 | 0.006 | 0.005 | 0.008 | 0.043 |
| CV | | % | 15.0 | 18.1 | 9.9 | 7.1 | 8.2 |
| AC | | % | 102 | 104 | 104 | 109 | 104 |
| Bias | | % | 2 | 4 | 4 | 9 | 4 |

[ . . . ] = Result rejected due to CV of duplicate measurements >30%

2.2.2.1 Lower and Upper Limit of Quantification

Lower limit of quantification (LLOQ) was determined at 0.02 μg/mL based on the inter-assay quality control data shown in Table 2 with lowest QC concentration with precision and accuracy <25% and 75-125%, respectively.

Upper limit of quantification (ULOQ) was determined at 0.5 μg/mL based on the inter-assay quality control data shown in Table 2 with highest QC concentration with precision and accuracy <20% and 80-120%, respectively.

1. A competitive assay method for assaying humanized antibody, Campath-1H (alemtuzumab), which binds to the CD52 cell membrane receptor, in a biological sample, said method comprising the steps of:
(a) obtaining a sample to be analyzed for the presence of the antibody;
(b) binding CD52 receptor containing cells or cell membrane preparations to a filter plate membrane;
(c) contacting the analyte antibody labeled with a detectable label and the test sample with the filter plate membrane, thus letting the labeled antibody and the antibody in the test sample compete for binding to cell membrane receptors in the filter plate membrane;
(d) washing unbound reagents through the filter plate membrane;
(e) detecting the presence of the label and determining the amount of Campath-1H (alemtuzumab) by referring to a calibration standard curve.

2. The method according to claim 1, wherein the labeled antibody is Campath-1H labeled with a fluorescent label.

3. The method according to claim 2, wherein the labeled antibody is Eu-labeled Campath-1H.

4. The method according to claim 1, wherein the biological sample is a serum, plasma, whole blood, cerebrospinal fluid or synovial fluid sample.

5. Use of the method according to any one of the preceding claims in pharmacokinetic studies or for monitoring purposes.

6. A test kit for assaying humanized antibody, Campath-1H (alemtuzumab), which binds to the CD52 cell membrane receptor, said kit comprising
a detectable label attached to the analyte antibody Campath-1H
a filter plate membrane
a (lyophilized or frozen) cell or cell membrane preparation,
in a suitable container.

7. The test kit according to claim 6, wherein the kit comprises Eu-labeled Campath-1H.
